(19) United States
(12) Patent Application Publication
(10) Pub. No.: US 2022/0340867 A1
(43) Pub. Date: Oct. 27, 2022

(54) METHOD FOR FABRICATION OF EXTRACELLULAR MATRIX-INDUCED SELF-ASSEMBLY AND FABRICATION OF ARTIFICIAL TISSUE USING SAME

(71) Applicant: AJOU UNIVERSITY INDUSTRY-ACADEMIC COOPERATION FOUNDATION, Suwon-si (KR)

(72) Inventors: Do Young PARK, Suwon-si (KR); Hee Woong YUN, Seoul (KR)

(73) Assignee: AJOU UNIVERSITY INDUSTRY-ACADEMIC COOPERATION FOUNDATION, Suwon-si (KR)

(21) Appl. No.: 17/763,936

(22) PCT Filed: Sep. 17, 2020
(86) PCT No.: PCT/KR2020/012585

§ 371 Date: Mar. 25, 2022

(30) Foreign Application Priority Data
Sep. 27, 2019 (KR) 10-2019-0119617

(51) Int. Cl. C12N 5/00; A61L 27/36; A61L 27/54; C12N 5/071

(52) U.S. Cl. CPC C12N 5/0068; A61L 27/3633; A61L 27/3683; A61L 27/54; C12N 5/0697; C12N 2533/54; C12N 2533/90

(57) ABSTRACT

The present invention relates to a method for fabrication of an extracellular matrix-induced self-assembly and to fabrication of an artificial tissue using same. The method for fabrication of an extracellular matrix-induced self-assembly comprise the steps of: (a) decellularizing and powdering a tissue-derived extracellular matrix (ECM); and (b) adding the decellularized extracellular matrix powder to cells and culturing the cells to form a cell-extracellular matrix powder self-assembly. Accordingly, the self-assembly has characteristics similar to those of extracellular matrix tissues and can be fabricated into three-dimensional artificial tissues 1 cm or greater in size, thus finding advantageous applications as a cell therapy product and an artificial tissue implant.

### TECHNICAL FIELD

The present invention relates to a method for fabrication of an extracellular matrix-induced self-assembly and fabrication of an artificial tissue using the same.

### BACKGROUND ART

Recently, tissue engineering has been attracting attention in order to regenerate organs or tissues damaged due to diseases or accidents. In such tissue engineering, biomaterials such as natural materials and synthetic polymers are used as supports for cell proliferation for morphological reconstruction of organs or tissues, and these are called scaffolds.

An artificial tissue can be produced by seeding cells or stem cells of a target tissue on the scaffold, culturing them in a suitable environment in vitro, and proliferating and differentiating into desired cells. Alternatively, there have been many reports of methods for regenerating a target organ by implanting the scaffold in which cells are seeded into a defect in an organ or tissue to be regenerated and differentiating them into cells expressing traits similar to those of the target tissue in vivo to proliferate the cells into a three-dimensional structure.

However, there have been problems in that, even if a bio-artificial organ is built using the scaffold, the physiological function of the organ cannot be sufficiently exhibited, or it is difficult to build the artificial tissue itself regardless of the presence or absence of the scaffold. In particular, as a carrier for seeding and transporting cells, most of the scaffolds are made of synthetic materials, and it is still difficult to control the biocompatibility and in vivo degradation and absorption of these synthetic biomaterials, which limits their clinical applications. In addition, non-uniform cell distribution during cell seeding and immune response due to malabsorption after transplantation are also issues to be solved.

On the other hand, there have been many reports on the development of tissue for transplantation using a scaffold-free engineering method without using the scaffold, which was considered an essential condition for tissue engineering. According to cell sheet engineering, announced in 1993 by Professor Okano of Tokyo Women's University in Japan, cultured cells can be used as a “sheet-shaped” tissue in which cells are interconnected by simply lowering the temperature of the cultured cells. In addition, if a high-density cell suspension is cultured using a rotating culture technique, cells form a spheroid with each other to form a spheroidal aggregate. This phenomenon has been reported to occur in fibroblasts and chondrocytes.

However, the existing scaffold-free tissue engineering method has several limitations. First, there is a limit to the size of the tissue obtained in an in vitro environment without blood vessel distribution, because supply of nutrients and oxygen that depends on diffusion is limited. In general, it is known that a distance of 300 μm or less is required for the survival of cells located inside through material exchange by diffusion. Therefore, it is usually difficult to manufacture artificial tissues with a diameter of 1 cm or more in the in vitro environment, and even if they can be manufactured, there is a limitation in that they are easy to be manufactured as necrosis of internal cells or non-homogeneous tissues. Second, the scaffold-free method requires a large amount of cells compared to the scaffold method on the basis of implants of the same size. Based on musculoskeletal chondrocytes, the scaffold free method produces a tissue with a size that is at least 100 times smaller than that of the scaffold method, and thus requires a large number of cells to reach a certain size. Third, the scaffold-free tissue engineering method requires additives to induce differentiation, such as growth factors, in the manufacture of target tissues, which may cause issues of increased cost and stability of the production process.

Therefore, there is a need for research on a method which requires no scaffold in tissue engineering and can fabricate an artificial tissue of 1 cm or more without the need for a separate differentiation-inducing additive.

### DISCLOSURE

### Technical Problem

An object of the present invention is to provide a method for fabrication of an extracellular matrix-induced self-assembly capable of fabricating an artificial tissue without the need for a separate scaffold and a differentiation inducing additive.

In addition, another object of the present invention is to provide a cell-extracellular matrix powder self-assembly, an artificial tissue, and an artificial organ fabricated by the method.

### Technical Solution

In order to achieve the above object, the present invention provides a method for fabrication of an extracellular matrix-induced self-assembly, the method including (a) decellularizing and powdering a tissue-derived extracellular matrix (ECM); and (b) adding the decellularized extracellular matrix powder to cells and culturing the cells to form a cell-extracellular matrix powder self-assembly.

Further, the present invention provides a cell-extracellular matrix powder self-assembly formed according to the method described above.

Furthermore, the present invention provides an in vitro matrix-derived artificial tissue formed according to the method described above.

In addition, the present invention provides an in vitro matrix-derived artificial organ formed according to the method described above.

### Advantageous Effects

According to the present invention, it is possible to form high-quality artificial tissues or artificial organs with uniform cell distribution capable of self-assembly into extracellular matrix-derived tissues without the need for a separate scaffold and differentiation-inducing additive by fabricating the extracellular matrix as powder, adding it to stem cells, and culturing the stem cells.

Further, it is possible to fabricate three-dimensional artificial tissues with a size of 1 cm or more only by controlling the concentration of the extracellular matrix powder added to the stem cells, and to fabricate the extracellular matrix-derived artificial tissues, which can be useful as a cell therapy product and an artificial tissue implant.

### BRIEF DESCRIPTIONS OF THE DRAWINGS

FIG. 1 shows the entire process of a method for fabrication of a cell-ECM powder self-assembly and its use as an implant of according to the present invention.

FIG. 2 shows forms of tissue-derived ECM powder (A) and analysis of particle size distribution (B).

FIG. 3 shows decellularization of various biological tissue ECM-derived powders (A) and differences in biochemical properties (B˜D).

FIG. 4 shows that ECM powders added to cells are cell-friendly (A-B), and that there is a difference in physiological activity depending on origin tissues (C-D).

FIG. 5 shows results of RT-PCR analysis of differences in differentiation-inducing patterns of stem cells according to the origin of different tissue ECM powders.

FIG. 6 shows that the size of the fabricated cell-ECM powder self-assembly can be adjusted according to the concentration of the added ECM-powder.

FIG. 7 shows that there are differences in the degree of differentiation depending on the origin of the ECM powder added as gross images and safranin-o staining images of the fabricated cell-ECM powder self-assembly.

FIG. 8 shows results after 4 weeks from subcutaneous injection of the cells and ECM powder into nude mice, and show that artificial tissues were not only naturally formed, but also formed with biochemical properties similar to those of origin tissues of the ECM powders according to the origin tissues.

### MODES FOR CARRYING OUT INVENTION

Hereinafter, the present invention will be described in detail.

The present inventors fabricated a variety of animal tissue extracellular matrix (ECM)-derived powders, added them to stem cells, and culture the stem cells, thereby forming self-assembled Cell-ECM powder construct without the need for separate scaffolds and differentiation-inducing additives. The present invention has been completed by finding out that, the self-assembled Cell-ECM powder construct have characteristics similar to those of extracellular matrix tissue, and it is possible to fabricate three-dimensional artificial tissues with a size of 1 cm or more, which can be used as a cell therapy product and an artificial tissue implant.

The present invention provides a method for fabrication of an extracellular matrix-induced self-assembly, including (a) decellularizing and powdering a tissue-derived extracellular matrix (ECM); and

(b) adding the decellularized extracellular matrix powder to cells and culturing the cells to form a cell-extracellular matrix powder self-assembly.

In the cell-extracellular matrix powder self-assembly formed above, the ECM-powder not only may act as a chemoattractant to attract cells, but also has a strong binding ability with cells, and has the ability to promote proliferation and differentiation. Since differentiation is induced according to the type of extracellular matrix tissue, various biomimetic structures can be fabricated. Therefore, the ECM-powder is effective for cell adhesion and proliferation, and in particular may have a great effect on the differentiation of stem cells into specific cells.

In step (a), the tissue-derived extracellular matrix may be any one selected from the group consisting of cartilage, small intestine, meniscus, ligament and tendonous tissue, but organs and tissues of all animals including humans or purified ECM material may be used.

In addition, in step (b), the cell is a stem cell, and may be an autologous allogeneic or heterogeneous stem cell, specifically may be any one selected from the group consisting of mesenchymal stem cells, embryonic stem cells, and dedifferentiated stem cells, but is not limited thereto.

According to an embodiment of the present invention, in the step (a), the decellularizing may be performed after cartilage and meniscus tissue powder is fabricated, but it is also possible to perform the decellularizing and the powdering before tissue powdering.

Further, the decellularized extracellular matrix powder in step (b) may be added at a concentration of 0.1 to 3 mg/ml, and the powder may be added to a culture solution or saline of the cells to be cultured, but is not limited thereto. Preferably, it may be added to the cells at a concentration of 1 to 2.5 mg/ml to culture the cells, but is not limited thereto.

Further, in step (b), the self-assembly may be formed in vitro or in vivo. In addition to a method of forming the cell-ECM self-assembly in vitro and transplanting it into a target tissue, the self-assembly may be formed in vivo by transplanting the ECM powder and cells immediately after mixing them.

Further, the cell-ECM powder self-assembly may be formed by inducing cell proliferation or cell differentiation in step (b).

Further, the present invention provides a cell-extracellular matrix powder self-assembly formed according to the self-assembly fabrication method described above.

Further, the present invention provides an in vitro matrix-derived artificial tissue formed according to the self-assembly fabrication method described above.

Furthermore, the present invention provides an in vitro matrix-derived artificial organ formed according to the self-assembly fabrication method described above.

Here, the cell-extracellular matrix powder self-assembly may be used as a cell therapy product as an active ingredient, and the cell therapy product may be directly inserted into a site to be treated using a syringe, inserted through surgery, or inserted through the circulatory system, but is not limited thereto.

In addition to inserting the self-assembly in vivo as described above, it may be formed in vivo by mixing the ECM powder according to the present invention and cells and immediately transplanting it to the site to be treated.

Here, the transplanting of the self-assembly or the ECM powder and cell mixture to the site to be treated as described above may include replacing damaged cells in a recipient tissue to heal tissue damage, or tissue rebuilding by forming a network with the recipient tissue, but is not limited thereto.

In addition, the disease to which the cell therapy product may be applied may be any one selected from the group consisting of autoimmune diseases, cardiovascular diseases, bone diseases, and neurological diseases, but is not limited thereto.

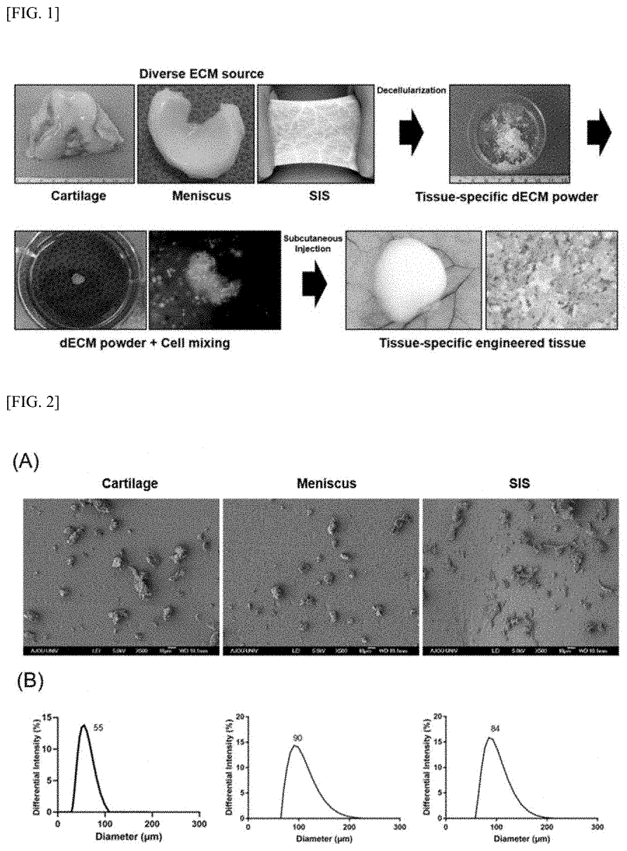

The present inventors found that, in the case of seeding stem cells in a culture dish and treating them with the ECM powder, spontaneous fusion occurred between the cells and the ECM powder to result in a single mass. It was confirmed that, this self-assembly phenomenon occurred in ECM-derived powders of various tissues such as cartilage, meniscus, and small intestinal submucosa and the fabricated stem cell-ECM powder self-assembly was induced to differentiate due to the biochemical characteristic of the derived-ECM powders without additional physiologically active factors. In particular, the size of the cell-powder self-assembly could be controlled depending on the amount of the ECM powder added, and it was possible to fabricate a homogeneous artificial tissue with a size of 1 cm or more. Based on the self-assembly phenomenon and differentiation-inducing property between these cells-ECM powder, it was confirmed that artificial tissues were formed when ECM powders derived from tissues of different origins and mesenchymal stem cells were injected subcutaneously and transplanted into nude mice. In addition, in the case that a biochemical analysis of the formed artificial tissue was performed, it could be confirmed that the artificial tissue was differentiated into the characteristics of the tissue from which the ECM powder was derived (FIG. 1).

Hereinafter, the present invention will be described in detail with reference to examples. These examples are merely for explaining the present invention in detail, and it would be evident to those skilled in the art to which the present invention pertains that the scope of the present invention is not limited by these examples.

### <Example 1> Fabrication of Various Decellularized Tissue-Derived ECM Powders

1. Decellularization of Porcine Cartilage Tissue Powder

Porcine articular cartilage was harvested from the knee joint, hip joint, and elbow joint using a surgical blade. The collected cartilage tissue was washed with DW and dried through a freeze dryer (Bondiro, Ilshinlab, Daejeon, Korea). Subsequently, the lyophilized tissue was obtained as a fine powder through a freezer mill (6870; SPEX, Metuchen, N.J., USA). Then, the tissue powder was treated with a low temperature buffer (10 mM Tris-HCl, pH 8.0) at room temperature for 12 hours, and then treated with Tris-buffered saline (TBS buffer) containing 0.1% sodium dodecyl sulfate at room temperature for 2 hours to perform decellularization. The decellularized cartilage powder was centrifuged at 10,000 RCF for 10 mM at 4° C. and washed 7 times with DW to remove detergent. Thereafter, the collected cartilage tissue powder was treated with Dnase buffer (100 U/ml, Elpis Biotech, Daejeon, Korea) at 4° C. for 12 hours to remove remaining genetic material. The final decellularized cartilage tissue powder was centrifuged with 10,000 RCF at 4° C. for 10 minutes and washed 7 more times with DW. The decellularized cartilage powder was freeze-dried and fabricated in a final powder form through a freezer mill. Then a powder having a size of 100 μm or less was obtained using a molecular sieve.

2. Decellularization of Porcine Meniscus Tissue Powder

Porcine meniscus was harvested from the knee joint using the surgical blade. The collected meniscus tissue was washed with DW and dried through the freeze dryer (Bondiro, Ilshinlab, Daejeon, Korea). Subsequently, the lyophilized tissue was obtained as a fine powder through the freezer mill (6870; SPEX, Metuchen, N.J., USA). Then, the tissue powder was treated with a low temperature buffer (10 mM Tris-HCl, pH 8.0) at room temperature for 12 hours, and then treated with Tris-buffered saline (TBS buffer) containing 0.1% sodium dodecyl sulfate at room temperature for 2 hours to perform decellularization. The decellularized meniscus powder was centrifuged at 10,000 RCF at 4° C. for 10 min and washed 7 times with DW to remove detergent. The collected cartilage tissue powder was treated with Dnase buffer (100 U/ml, Elpis Biotech, Daejeon, Korea) at 4° C. for 12 hours to remove residual genetic material. The final decellularized cartilage tissue powder was centrifuged with 10,000 RCF at 4° C. for 10 minutes and washed 7 more times with DW. The decellularized cartilage powder was freeze-dried and fabricated in a final powder form through the freezer mill. Then, a powder having a size of 100 μm or less was obtained using the molecular sieve.

3. Decellularization of Pig Small Intestine Submucosal Tissue (SIS) Powder

Fabrication of decellularized small intestine submucosal tissue (SIS) mechanically removed the tunica mucosa, tunica serosa and tunica muscularis from the small intestine tissue, and left the tunica submucosal layer and basilar part. Decellularization and disinfection were performed by treatment with 0.1% acetic acid containing 4% ethanol at 300 rpm for 2 hours at room temperature. The decellularized SIS was then washed 7 times with PBS. The washed SIS was freeze-dried and pulverized to fabricate a final powder form through the freezer mill. Then, a powder having a size of 100 μm or less was obtained using the molecular sieve.

### <Example 2> Physical Morphology and Particle Size Analysis of Decellularized ECM Powders

1. Morphological Analysis of Decellularized ECM Powders

The morphology of freeze-pulverized porcine cartilage powders was analyzed using a scanning electron microscope. The ECM powders pulverized in Example 1 were dehydrated with ethanol and dried, and the size and shape of the powders were observed with an electron microscope (JEOL, JSM-6380, Japan; 20 KV).

As a result, it was determined that the average size of the decellularized extracellular matrix (ECM) powders was about 10-200 μm (FIG. 2A).

2. Particle Size Distribution Analysis of Decellularized ECM Powders

The decellularized ECM powders were mixed with DW at a concentration of 100 μg/ml, and the particle size distribution was measured through a dynamic light scattering method (ELSZ-2000, Otsuka Electronics, Osaka, Japan).

As a result, the ECM powder particle size distribution was measured to be about 10-200 μm, and it was determined that the cartilage ECM powder had a diameter of about 55 μm, the meniscus ECM powder had a diameter of about 90 μm, and the SIS ECM powder had a diameter of about 84 μm (FIG. 2B).

### <Example 3> Analysis of Biochemical Properties of Decellularized ECM Powders

1. Analysis of DNA Content of Decellularized Tissue-Derived ECM Powders

In order to check whether the ECM powder subjected to the decellularization process was decellularized, the amount of remaining dsDNA was quantified through picogreen assay (p11496, ThermoFisher Scientific, USA).

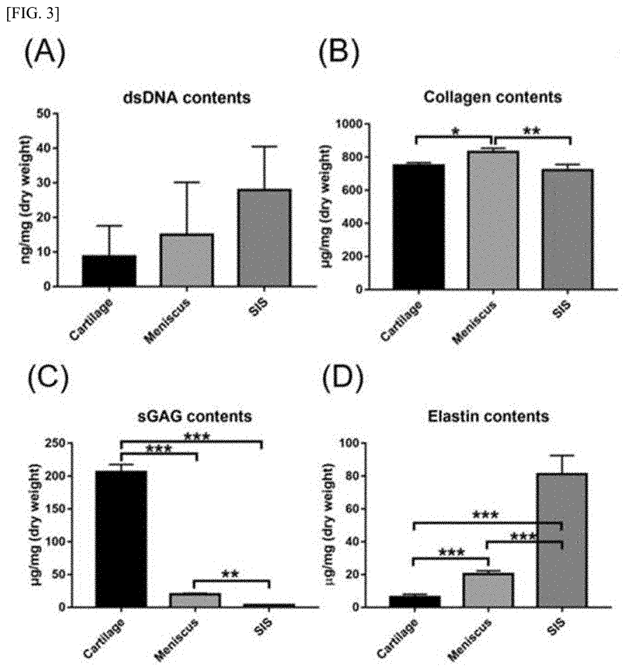

Typically, the amount of dsDNA allowed for transplantation in vivo is 50 ng or less per 1 mg of unit tissue, so it was confirmed that decellularization was successfully performed in all of cartilage, meniscus, and SIS ECM (FIG. 3A).

2. Analysis of Component Contents of Tissue-Derived ECM Powders

In order to analyze differences in the contents of components according to the origin of the tissue, the contents of collagen, sulfated glycosaminoglycan (sGAG), and elastin were analyzed.

Collagen was measured using 51000 (Biocolor, UK), sGAG was measured using B1000 (Biocolor, UK), and elastin was measured using F2000 (Biocolor, UK).

As a result, it was determined that collagen occupied the highest proportion in the meniscus (FIG. 3B), and sGAG occupied the highest proportion in the cartilage (FIG. 3C). In addition, it was determined that elastin had a significantly high content in the small intestine submucosal tissue (FIG. 3D).

### <Example 4> Analysis of Cell-Specific Properties of Decellularized ECM Powders

1. Analysis of Mesenchymal Stem Cell Affinity of Decellularized ECM Powders

In order to evaluate differences in the effect on cell behavior according to the origin of the tissue, analysis on cell proliferation, survival, migration, and adhesion was performed. The ECM powder of each tissue was added at a concentration of 1 mg/ml to a culture dish with a diameter of 6 cm in which human synovial-derived mesenchymal stem cells (hMSCs) were seeded with 4×10⁶ cells and was cultured for 1, 4, 7, 10, and 14 days at 37° C. under 5% CO₂ condition.

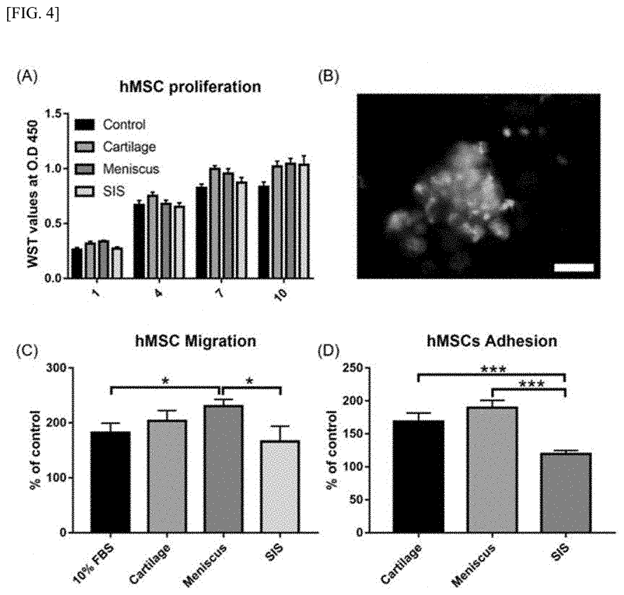

The proliferation of hMSC was analyzed by WST assay, and as a result, it was determined that the proliferation increased in the case that the ECM powder was added compared to the control with nothing added (FIG. 4A). In addition, it was determined that the hMSC showed more affinity to the ECM powder surface by showing a tendency of migrating from the surface of the culture dish to the ECM powder particle surface to adhere thereto. It was confirmed that the adhesion between the cell and the ECM powder was further promoted as the culture period progressed and the cells and ECM powder were fused to form one large particle, and that the cells did not die and survived well even on the 14th day of culture through LIVE DEAD assay (FIG. 4B).

2. Analysis of Mesenchymal Stem Cell Mobility of Decellularized ECM Powder

Boyden chamber assay was performed to evaluate whether the ECM powder can promote cell migration with biochemical chemotaxis.

As a result, in the case of hMSC, it was confirmed that the meniscus ECM powder attracted the most cells, and the number was significantly higher than that of a cell culture medium containing 10% FBS (FIG. 4C).

3. Analysis of Mesenchymal Stem Cell Adhesion Ability of Decellularized ECM Powders

Cell adhesion was evaluated to observe the difference in affinity between hMSC and ECM surfaces according to the origin of each tissue ECM powder.

For cell adhesion evaluation, each tissue ECM powder was mixed with an agarose gel at a concentration of 1 mg/ml and coated on a culture dish. Then, hMSC was inoculated and the culture dish was washed twice with PBS 2 hours later, and the cells attached to the agarose were stained with Calcein AM to measure the absorbance.

As a result, it was confirmed that more hMSCs were attached to the surface to which the ECM powder was added in the case that agarose with nothing mixed in was used as a control group, and that in particular, the adhesion was increased at a significantly higher level in the meniscus ECM powder compared to other groups (FIG. 4D).

### <Example 5> Analysis of In Vitro Mesenchymal Stem Cell Differentiation Induction of ECM Powders

1. Analysis of Mesenchymal Stem Cell Differentiation Induction of ECM Powders According to Tissue Origins

To evaluate the effect of the addition of the ECM powders on the differentiation of hMSCs according to the origins of the tissues, the expression of type 1 and type 2 collagen, aggrecan, and SOX-9 was analyzed through RT-PCR.

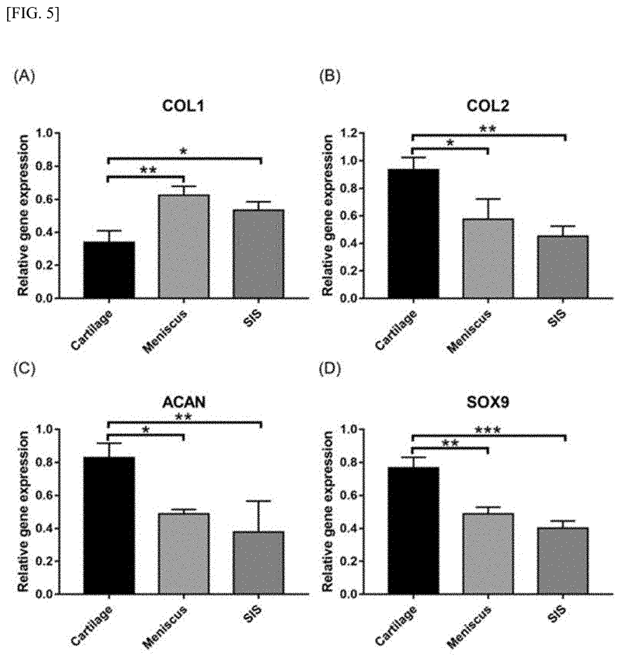

As a result, the meniscus and the small intestine submucosa significantly increased the expression of the type 1 collagen gene compared to the cartilage ECM powder-added group. In addition, it was confirmed that the addition of the cartilage ECM powder significantly increased the expression of the type 2 collagen of hMSC compared to other groups, and also significantly increased the expression of aggrecan and SOX 9, which are other cartilage tissue markers (FIG. 5).

Therefore, it was confirmed that the ECM powder induced cell differentiation according to the characteristics of the tissue of origin.

### <Example 6> In Vitro Cell-ECM Powder Self-Assembly Fabrication

1. In Vitro Cell-ECM Powder Self-Assembly Fabrication and Size Control Analysis

A cell-ECM powder self-assembly was fabricated in the following process.

hMSCs were seeded with 4×10⁶ cells in a 6 cm diameter culture dish and cultured at 37° C. under 5% CO₂ condition for 3 days. Thereafter, each of the ECM powders fabricated according to Example 1 was suspended in a cell culture medium (alpha-MEM) containing 10% FBS at a concentration of 1 mg/ml, and then it was put into a cell culture medium and cultured at 37° C. for 1 day under 5% CO₂ condition. When the cell-ECM powder start to fuse, the cell-ECM powder self-assembly was carefully separated using a cell scraper and transferred to a 50 ml tube containing 5 ml of cell culture medium to be cultured, and then the cell culture medium was replaced once every 3 days.

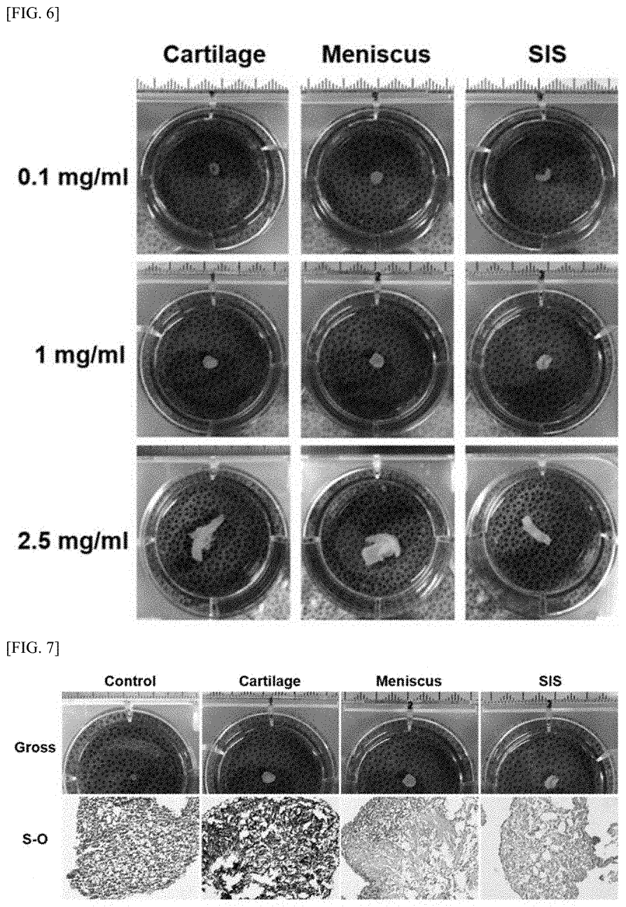

As a result, it was observed that the cell-ECM powder self-assembly formed a more condensed form after about 3 days to a week after culturing in the 50 ml tube. In addition, it was determined that the size of the self-assembly increased as the concentration of ECM powder increased, and in the case of being treated with the ECM powder at a concentration of 2.5 mg/ml, the self-assembly with a diameter up to 1 cm could be fabricated (FIG. 6).

2. Gross Evaluation and Histological Analysis of In Vitro Cell-ECM Powder Self-Assembly

It was visually confirmed that the cell-ECM powder self-assembly treated with the ECM powder at a concentration of 1 mg/ml could be fabricated in a shape close to a spheroid. As a result of histological observation by Safranin-O staining, it was confirmed that the cell-ECM powder self-assembly formed a homogeneous internal distribution. In addition, it could be observed that the cells were homogeneously attached to the ECM powder from histological observation, and it was confirmed that there was a difference in the degree of Safranin-O staining depending on the ECM powder origin tissue (FIG. 7).

### <Example 7> Analysis of Artificial Tissue Formation In Vivo of Cell-ECM Powder Self-Assembly

1. Gross and Histological Analysis of In Vivo Cell-ECM Powder Self-Assembly Tissue

In order to evaluate an in vivo forming ability of the cell-ECM powder self-assembly, hMSCs at a concentration of 1×10⁶ cells and the ECM powder at a concentration of 1 mg/ml were suspended in saline, and 100 ul each was injected subcutaneously to nude mice. Four weeks after the subcutaneous injection, histological evaluation was performed through visual observation and H&E staining, Safranin-O staining, and collagen type I (COL I) and collagen type II (COL II) staining to evaluate the degree of tissue formation and differentiation by sacrificing the nude mice.

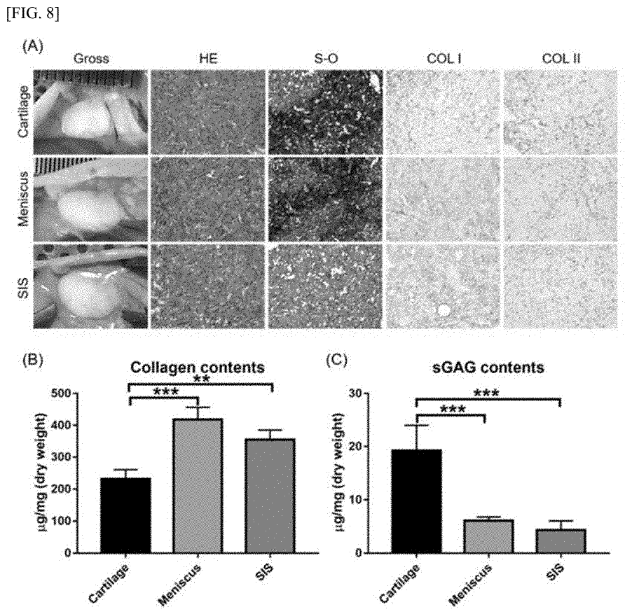

As a result, it was visually observed that a compact and homogeneous artificial tissue was formed at the location where the cell-ECM powder suspension was injected. Further, as a result of the H&E staining, it was determined that the cytoplasm was formed homogeneously and the distribution of cells was also homogeneous. In addition, it was confirmed that the patterns of the Safranin-O staining and the expression of the collagen type similar to those of the origin tissue of the ECM powder were observed in the artificial tissue (FIG. 8A).

2. Component Analysis of In Vivo Cells-ECM Powder Self-Assembly Tissue

Collagen and sGAG contents were quantitatively evaluated to evaluate the component content analysis of artificial tissues formed in vivo according to the origins of the ECM powders.

As a result, it was confirmed that as in FIGS. 3B and 3C of <Example 3>, the collagen content was measured significantly higher than those of other groups in the meniscus, and the sGAG content showed a significantly high quantitative value in the cartilage tissue (FIG. 8B).

Therefore, it was confirmed that the ECM-powder not only could act as a chemoattractant to attract cells, but also had the strong binding ability with cells and the ability to promote proliferation and differentiation. Due to these results, it has been determined that a fusion action occurs between the cells and the ECM powder eventually to fabricate a single self-assembly and thus the ECM-derived artificial tissue can be formed.

1. A method for fabrication of an extracellular matrix-induced self-assembly, the method comprising:
(a) decellularizing and powdering a tissue-derived extracellular matrix (ECM); and
(b) adding the decellularized extracellular matrix powder to cells and culturing the cells to form a cell-extracellular matrix powder self-assembly.

2. The method according to claim 1, wherein the tissue-derived extracellular matrix in (a) is any one selected from the group consisting of cartilage, small intestine, meniscus, ligament, and tendinous tissue.

3. The method according to claim 1, wherein the cell in (b) is a stem cell.

4. The method according to claim 3, wherein the stem cell is any one selected from the group consisting of a mesenchymal stem cell, an embryonic stem cell, and a dedifferentiated stem cell.

5. The method according to claim 1, wherein the decellularized extracellular matrix powder is added at a concentration of 0.1 to 3 mg/ml in (b).

6. The method according to claim 1, wherein the self-assembly is formed in vitro or in vivo in (b).

7. The method according to claim 1, wherein the cell-extracellular matrix powder self-assembly is formed by inducing cell differentiation or cell proliferation in (b).

8. A cell-extracellular matrix powder self-assembly formed according to the method of claim 1.

9. An in vitro matrix-derived artificial tissue formed according to the method of claim 1.

10. An in vitro matrix-derived artificial organ formed according to the method of claim 1.
